(19) United States
(12) Patent Application Publication
(10) Pub. No.: US 2015/0238371 A1
(43) Pub. Date: Aug. 27, 2015

(54) RECOVERING PACKAGE FOR BULKY ARTICLES

(71) Applicant: Kimberly-Clark Worldwide, Inc., Neenah, WI (US)

(72) Inventor: Autumn Joy Price, Appleton, WI (US)

(73) Assignee: Kimberly-Clark Worldwide, Inc., Neenah, WI (US)

(21) Appl. No.: 14/191,810

(22) Filed: Feb. 27, 2014

(51) Int. Cl. A61F 13/551; B65D 85/18; B65D 33/14; B65D 33/18; B65D 21/08; B65D 33/02

(52) U.S. Cl. CPC A61F 13/5511; B65D 21/08; B65D 33/02; B65D 33/14; B65D 33/18; B65D 85/18; A61F 2013/55195

(57) ABSTRACT

A new package for storing bulky articles formed from a bag and a housing portion. The bag sealed is together at a pair of side edges and has a base portion and a top portion. The bag has a depth and a first width at the base portion forming a cavity in the base portion. The housing portion is stored within the cavity formed by the base portion of the bag, the housing portion having a base panel, a front panel, a back panel, a first side and a second side. Both the first side and second side are formed from a first side portion and a second side portion. The first side portion overlaps the second side portion and is adapted to move relative to the second side portion to enable a change in a depth of the housing portion.

### BACKGROUND

People rely on disposable absorbent garments in their everyday lives, including such garments as adult incontinence products, enuresis pants, training pants, and diapers. Many manufacturers seek to better meet the needs of users of such products. With certain products, such as adult incontinence underwear and enuresis pants, it is important that the garments look and feel as much as possible like “regular” underwear to promote an improved sense of normalcy to the wearer who suffers from incontinence or enuresis. Additionally, purchasers and users of such products are frequently embarrassed about their condition and about having to purchase products to deal with their incontinence or enuresis condition.

Currently, the most common method for obtaining incontinence and enuresis underwear is by purchasing relatively large bags in retail stores. Such conventional packages of incontinence and enuresis underwear are opaque or mostly opaque, which some purchasers may perceive as overly “diaper-like” or too strongly connoting the presence of a personal care absorbent product directed to a urinary condition. Such conventional packaging bears little resemblance to the packaging in which durable clothing is sold. There is a need for a package of incontinence or enuresis underwear that better resembles a package of durable underwear so as to improve the feeling of normalcy for the purchaser/user. There is also a need for a package of incontinence or enuresis underwear that allows the potential purchaser to see selected features of the underwear, such as, for example, cloth-like material used to make the underwear, elastic waistbands, and back-label indicators.

Attempts have been made in the art to provided windowed packages to allow consumers to inspect the absorbent garments contained within the sealed packages prior to purchase. However, the prior art does not optimally provide for targeted display and/or obscurement of particular features of pants, particularly with larger underwear such as incontinence and enuresis underwear, some of which frequently require multiple folds in both directions to efficiently configure the underwear for commercial packaging and sale.

Durable clothing and other bulky goods are often sold in a package system that combines a rectangular carton inside a poly bag to allow for displaying targeted displays of features while still maintaining the products in the correct position in the package. Many bags also require a header banner for branding and hanging the package on retail hooks.

A packaging structure is needed that can be produced that allows for resembles a package of durable underwear so as to improve the feeling of normalcy for the purchaser/user.

### SUMMARY

To better meet the above-described unmet needs in the art, a new package is disclosed herein. Generally, the package is formed from a bag and a housing portion. The bag sealed is together at a pair of side edges and has a base portion and a top portion. The bag has a depth and a first width at the base portion forming a cavity in the base portion.

The bag also includes a header strip formed across the top edge of the bag. The housing portion is stored within the cavity formed by the base portion of the bag, the housing portion having a base panel, a front panel, a back panel, a first side and a second side. Both the first side and second side are formed from a first side portion and a second side portion. The first side portion overlaps the second side portion and is adapted to move relative to the second side portion to enable a change in a depth of the housing portion.

In some embodiments, the first side portion and second side portion are not permanently attached to each other to enable the first side portion and second side portion to move relative to each other. By allowing the first side portion and second side portion to move relative to each other, the depth of the housing portion at a top edge of the housing portion is able decreases when a force is placed upon it and recovers to the original housing portion depth when the force is removed. This is done without any evidence of the package being handled or any damage to the package. In desirable embodiments, the depth of top edge of the housing portion decreases about 0.5 inches without noticeable damage of the package.

In desirable embodiments, the housing portion has an opening at a top of the housing portion for displaying and removing articles. A feature of the articles stored within the housing extends through the opening of the housing portion and is visible to a consumer.

In other desirable embodiment, the bag includes a header strip. In some embodiments, a hole in the header strip enabling the package to be hung on a pin or rod on a display rack in a first shelf configuration. The header strip has a header strip width that may desirably be substantially similar in width to the first width of the base portion of the package. In other embodiments, a header strip width that may be greater or smaller in width than the first width of the base portion of the package.

Use of a housing portion within the bag allows for two types of storage configurations of the packaging. A hole in the header strip enables the package to be hung on a pin or rod on a display rack in a first shelf configuration. In this embodiment, the housing portion would have a flat bottom enabling the package to be placed on a shelf in a second shelf configuration. Having a package with two types of shelf configuration provides more flexible position on the store shelf.

### DESCRIPTION OF THE DRAWINGS

FIG. 1 representatively illustrates a front plan view of the package shown in the embodiment of FIG. 1 including a housing portion and articles disposed therein.

FIG. 2 representatively illustrates a side plan view of the package shown in the embodiment of FIG. 1 including a housing portion and articles disposed therein.

FIG. 3 representatively illustrates a view of the housing portion utilized in the package, the housing portion laid out longitudinally in a laid-flat condition, prior to the folding the panels to provide the housing portion of FIG. 1.

### DETAILED DESCRIPTION

Reference to the Figures shall be made in describing various aspects and embodiments of the invention. It should be noted that the embodiments depicted in the Figures and described herein are merely representative examples of the pants and package of the invention. The various aspects and embodiments of the present invention are suitable for use with any goods. In some embodiments, adult incontinence pants, prefastened disposable diapers, disposable swim pants, disposable training pants, disposable enuresis garments, and the like may be used with the package.

Generally, a new package for storing bulky goods is disclosed. Generally, the package is formed from a bag and a housing portion. The bag sealed is together at a pair of side edges and has a base portion and a top portion. The bag has a depth and a first width at the base portion forming a cavity in the base portion. The bag also includes a header strip formed across the top edge of the bag. The housing portion is stored within the cavity formed by the base portion of the bag, the housing portion having a base panel, a front panel, a back panel, a first side and a second side. Both the first side and second side are formed from a first side portion and a second side portion. The first side portion overlaps the second side portion and is adapted to move relative to the second side portion to enable a change in a depth of the housing portion. The construction of the housing portion allows for the package to be squeezed by a consumer to be able to feel the contents of the interior package.

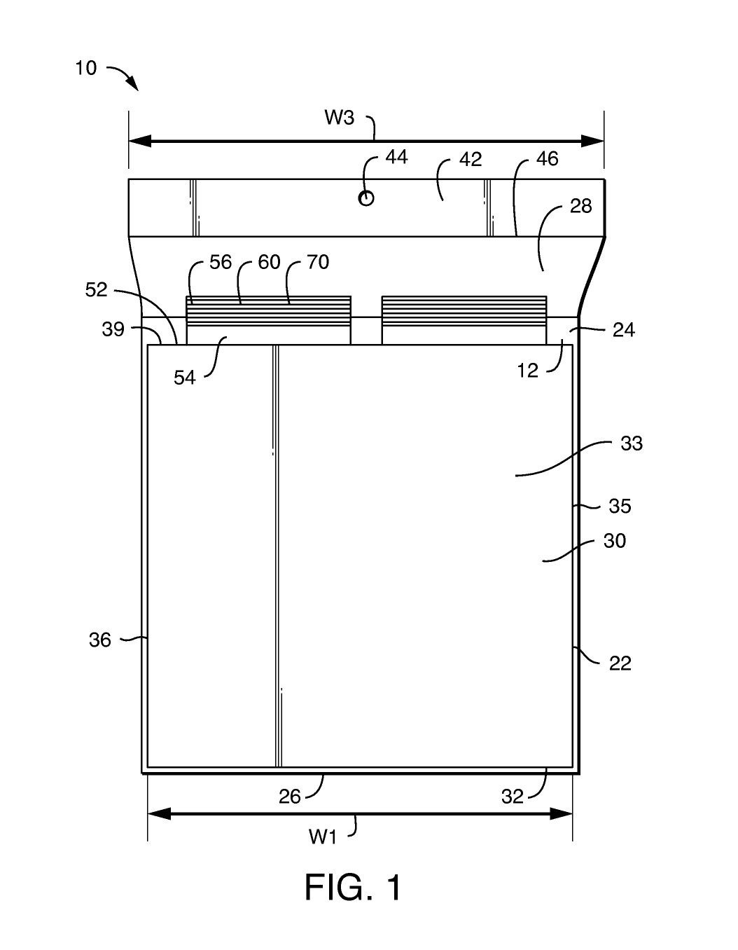

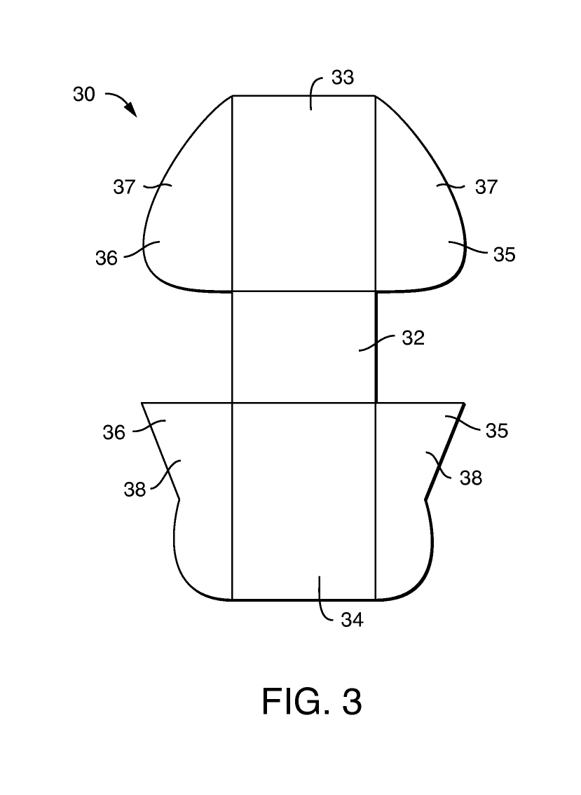

Turning to FIGS. 1-3, a package 10 is constructed from bag 12 and an interior package. The bag may be formed a tube from at least one material web and sealing tube at a pair of side edges. As illustrated, the bag is formed from a first web 14 and a second web 16 sealed together at a pair of side edges 18, 20. The first and second web 14, 16 may be sealed at the side edges 18, 20 by any means known to those skilled in the packaging art. For example, in one embodiment, the first and second web 14, 16 will be sealed with pinch seal, or an adhesive seal. When sealing with an adhesive, a hot melt adhesive is preferably used for sealing the side edges of the web. Ultrasonic or heat sealing may also be used to seal the side edges 18, 20 of the first and second web 14, 16 with or without a pinch seal and with or without adhesive. In addition, any combination of sealing techniques may also be used to seal the side edges 18, 20 of the first and second web 14, 16 together.

In exemplary embodiment, each of the first and second web 14, 16 may be formed from a durable, flexible material suitable for such containment and storage of materials. Examples of suitable materials for the first web and second web include, but are not limited to paper, aluminum foil, metalized films, coated films, printed films, co-extruded films, polyester films, polyolefin based films, white polyolefin based films, polyamide based films, copolymer films, multi-layer polymer films and films containing various polymer blends. Preferably, the first web and second web is polyolefin based, such as a monoweb polymer. One desirable embodiment would be polypropylene. Other examples include polyethylene and blends of polypropylene and polyethylene and separate layers of polymers, and other intermediate extrusion layers can be utilized with the separate woven polypropylene layer and the propylene outer layer. These polymers can be blended in combination or used in separate, distinct sheets.

In some embodiments, the first web 14 and second web 16 are formed from the same materials. In other embodiments, the first web 14 and second web 16 are formed from different materials.

The constructed bag 12 includes a base portion 22 having a depth D1 and a first width W1 at the base portion 22 forming a cavity 24 between the first web 14 and second web 16. Once filled with the contents of the package 10, the bottom edges of the first and second web 14, 16 of the bag 12 may be folded together to provide a bottom 26 to the bag 12.

The bag 12 also includes a top portion 28. The top portion 28 of the bag 12 extends from a top portion transition point 30 adjacent the base portion 22 of the bag 12 to a top edge 46. The bag 12 is sealed at the top edge 46. The first and second webs 16, 18 may be sealed at the top edge 46 by any means known to those skilled in the packaging art. For example, in one embodiment, the web will be sealed with pinch seal, or an adhesive seal. When sealing with an adhesive, a hot melt adhesive is preferably used for sealing the side edges of the web. Ultrasonic or heat sealing may also be used to seal the side edges of the bag with or without a pinch seal and with or without adhesive. In addition, any combination of sealing techniques may also be used to seal the top edge 46 of the first and second web 14, 16 together.

In desirable embodiments, the first and second webs 14, 16 forming the bag 12 are transparent materials, allowing a consumer to see the contents of the package 10 for inspection. In other embodiments, the first and second webs 14, 16 are opaque.

Adjacent the top edge 46 of the bag 12 is an integral header strip 42. In some embodiments, a hole 44 in the header strip 42 enabling the package 10 to be hung on a pin or rod on a display rack in a first shelf configuration. The header strip has a header strip width W3 that may desirably be substantially similar in width to the first width W1 of the base portion 22 of the package 10. In other embodiments, a header strip width W3 that may be greater or smaller in width than the first width W1 of the base portion 22 of the package 10.

As illustrated, the package 10 includes a housing portion 30 to store the articles. The housing portion 30 includes a base panel 32, a front panel 33, a back panel 34, a first side 35 and a second side 36. Each of the sides 35, 36 are formed from a first side portion 37 and a second side portion 38. The first side portion 37 overlapping the second side portion 38 and is adapted to move relative to the second side portion 38 to enable a change in a depth D1 of the housing portion 50. The first side portion 37 and second side portion 38 are not permanently attached to each other to enable the first side portion 37 and second side portion to move relative to each other.

By allowing the first side portion 37 and second side portion 38 to move relative to each other, the depth D1 of the housing portion 50 at a top edge 39 of the housing portion 50 is able decreases when a force is placed upon it and recovers or bounces back to the original housing portion depth D1 when the force is removed. This is done without any evidence of the package being handled or any damage to the package. In desirable embodiments, the depth of top edge 39 of the housing portion 50 decreases about 0.5 inches without noticeable damage of the package. Allowing the package to temporarily deform and recover allows for a consumer to feel the contents of the package and still look presentable on shelf.

The housing portion 30 includes a housing depth, housing width, and housing height, the housing portion having an opening 52 at its top. The housing portion 30 of the package 10 may be formed of any suitable packaging material known to those skilled in the art, such as, for example, paper, plastic film, paperboard, corrugated board, flexible polymeric material, semi-rigid plastic, or combinations thereof. In one embodiment, the housing portion 30 at least in part comprises a carton, such as a paperboard carton. The housing portion 30 may optionally include one or more inserts disposed within the housing portion 30, such as a paperboard insert.

In a desirable embodiment, the housing portion 30 has an opening 52 through a top panel of the housing portion 30 for displaying an article or good. The articles or goods 54 are positioned within the housing portion 30 such the at least one article 54 extends above the height of the housing portion 30, and such that at least a feature 56 of the article 54 is visible through the transparent bag 12. For example, a feature 56 of the articles 54 extends through the opening 52 of the housing portion 30 and is visible to a consumer.

As illustrated in the figures, the package 10 may further include a plurality of articles disposed within the housing portion 30. In particular embodiments, the articles may include disposable absorbent pants or durable absorbent pants for use with disposable absorbent pads. Examples of these types of articles suitable for incorporation into particular embodiment of the present invention include those disclosed in U.S. Application Ser. No. 13/547,974 filed by Evenson et al. on Jul. 12, 2012, U.S. application Ser. No. 13/933,235 filed by Vignali et al. on Jul. 2, 2013, and U.S. application Ser. No. 13/933,260 filed by Evenson et al. on Jul. 2, 2013, the contents of each of which is hereby incorporated by reference to the extent consistent herewith. Examples of disposable absorbent pants having certain aspects suitable for incorporation into particular embodiments of the present invention include those disclosed in U.S. Pat. No. 5,745,922 issued May 5, 1998 to Rajala et al., U.S. Pat. No. 6,240,569 issued Jun. 5, 2001 to Van Gompel et al., U.S. Pat. No. 6,702,798 issued Mar. 9, 2004 to Christoffel et al., and U.S. Pat. No. 7,604,624 issued Oct. 20, 2009 to Veith et al., the contents of each of which is hereby incorporated by reference to the extent consistent herewith. Note that the disposable absorbent pants could be provide in a permanently “closed” (i.e., pull-on style) configuration, a releasably and refastenably “closed” configuration, or an “open” (i.e., non-prefastened) configuration—any of which could be used in conjunction with the various embodiments of the present invention. While illustrated as a garment, any object or article requiring the package to have bulk may be stored within the package described herein.

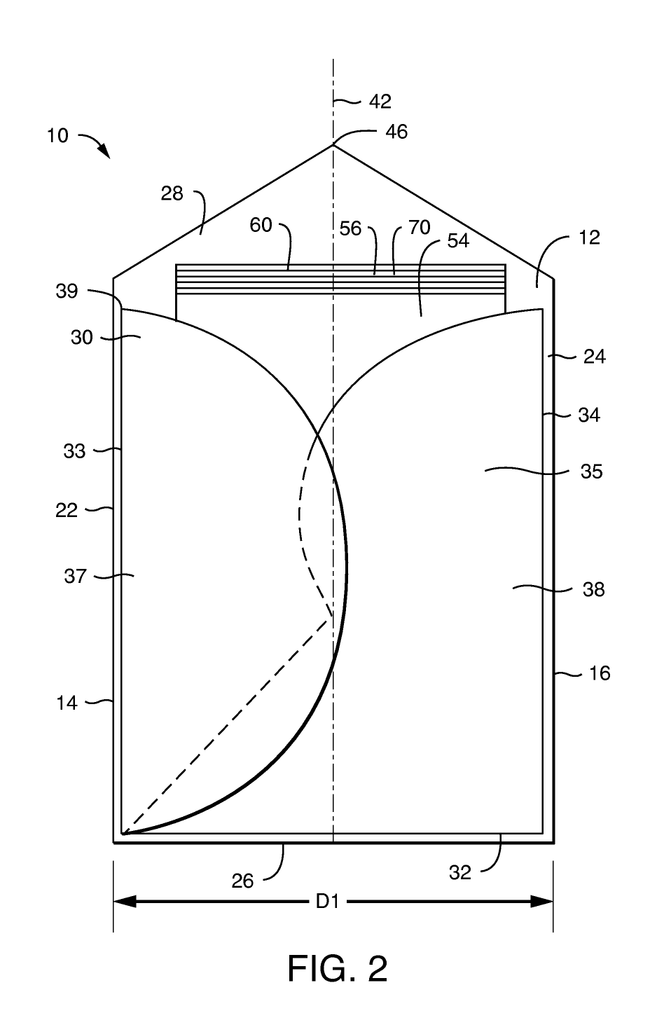

Referring to FIGS. 1-2, in particular embodiments at least one garment 60, such as at least two pants, of the plurality of articles is positioned within the housing portion 30 such the at least one garment 60 extends above the height of the housing portion 30, and such that at least a portion of the a feature 56 of the garment 60 is visible through the transparent bag. In this way, it can in certain embodiments be possible to highlight for a consumer the waistband region 70 of the garment or article. The feature 56 of the garments 60 that is illustrated through the package is selected from cloth-like material used to make the underwear, elastic waistbands, and back-label indicators. Without intending to be limiting, the present inventors have discovered that these particular dimensional relationships can in particular embodiments optimize the balance between displaying desired features 56 of the product through the package bag 12.

Allowing the package to temporarily deform and recover allows for a consumer to feel the article 60 stored within the package and still look presentable on shelf. As a result, the package resembles a package of durable underwear so as to improve the feeling of normalcy for the purchaser/user.

To illustrate the ability of the package to temporarily deform and recover, weights weighing 16 pounds are placed on a package 10 containing articles. This amount of weight was utilized to represent the typical force a consumer would use to squeeze the package to gain a better understanding of the product and the materials the product. The weight is applied at a midpoint of the front panel 33 of the housing portion 30 approximately 1 inch below the top edge 39 of the housing portion 50. The depth D1 of the housing portion 50 at its top edge 39 was measured prior to placing the weight onto the housing portion, while the weight is being applied to the package, and after the weight is removed and the package is allowed to relax. This amount of weight caused the depth D1 of the top edge 39 of the housing portion 50 to decrease about 0.5 inches. After the weight was removed, the depth D1 returned to normal without any damage to the product.

The housing portion 30 may also be used to store components of the goods that are undesirable for the consumer to see or require more discretion. For example, in the type of product with having both a durable absorbent pants for use with disposable absorbent pads, the absorbent pads may be stored within the housing and not visible to a consumer. If stored within the housing portion 30, the absorbent pads cannot be seen and provide more discretion to a consumer who purchases them at the store normalizing the purchase and providing a better consumer experience.

In addition, use of a housing portion 30 within the bag 12 allows for two types of storage configurations of the packaging. A hole in the header strip enables the package to be hung on a pin or rod on a display rack in a first shelf configuration. In this embodiment, the housing portion 30 would have a flat bottom enabling the package 10 to be placed on a shelf in a second shelf configuration. Having a package 10 with two types of shelf configurations provides more flexible positioning on the store shelf.

It will be appreciated that details of the foregoing embodiments, given for purposes of illustration, are not to be construed as limiting the scope of this invention. Although only a few exemplary embodiments of this invention have been described in detail, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. A package for storing goods, the package comprising:
a bag sealed together at a pair of side edges, the bag having a base portion and a top portion, the bag having a depth and a first width at the base portion forming a cavity in the base portion, and a header strip formed across the top edge of the bag; and
a housing portion stored within the cavity formed by the base portion of the bag, the housing portion having a base panel, a front panel, a back panel, a first side and a second side, wherein the first side and second side are formed from a first side portion and a second side portion, the first side portion overlapping the second side portion and adapted to move relative to the second side portion to enable a change in a depth of the housing portion.

2. The package of claim 1 wherein a depth of the housing portion at a top edge of the housing portion decreases when a force is placed upon it and recovers to the original housing portion depth when the force is removed.

3. The package of claim 2 wherein the depth of top edge of the housing portion decreases about 0.5 inches without destruction of the package.

4. The package of claim 1 wherein the bag is formed from a first web and second web sealed together at the side edges.

5. The package of claim 1 wherein the housing portion is selected from paper, plastic film, paperboard, corrugated board, flexible polymeric material, semi-rigid plastic, or combinations thereof.

6. The package of claim 1 wherein the housing portion has an opening through a top panel of the housing portion for placement of articles,

7. The package of claim 6 wherein a feature of the articles extends through the opening of the housing portion and is visible to a consumer.

8. The package of claim 7 further wherein the article comprises at least a first pant of positioned within the housing portion such that the first pant extends through the opening, and such that at least a portion of a waistband region of the first pant is visible through the bag.

9. The package of claim 7 wherein the feature of the articles is selected from cloth-like material used to make the underwear, elastic waistbands, color, printing and back-label indicators.

10. The package of claim 1 further comprising a hole in the header strip enabling the package to be hung on a pin or rod on a display rack in a first shelf configuration.

11. The package of claim 10 wherein the housing portion having a flat base panel enabling the package to be placed on a shelf in a second shelf configuration.

12. The package of claim 1 wherein the first side portion and the second side portion are not attached.

13. A package for storing an article, the package comprising:
a housing portion having a first side and a second side, wherein the first side and second side are formed from a first side portion and a second side portion, the first side portion overlapping the second side portion and able to move in a relative to the second side portion, the housing portion having an opening through a top of the housing portion;
a bag formed from a first web and a second web sealed together at a pair of side edges, the bag having a base portion and a top portion, the bag having a bag depth and a first bag width at the base portion forming a cavity between the first web and second web for storing the housing portion;
a header strip formed across the top edge of the bag having a header strip width; and
wherein the article stored within the housing extends through the opening of the housing portion and is visible to a consumer in the top portion of the bag.

14. The package of claim 13 wherein a depth of the housing portion at a top edge of the housing portion is able to deflect inward when a force is placed upon it and recovers to the original housing portion depth when the force is removed.

15. The package of claim 13 wherein the depth of the top edge of the housing portion deflects about 0.5 inches without destruction of the package.

16. The package of claim 13 wherein the housing portion is selected from paper, plastic film, paperboard, corrugated board, flexible polymeric material, semi-rigid plastic, or combinations thereof.

17. The package of claim 13 wherein the housing portion has an opening at the top of the housing portion for displaying articles, wherein a feature of the articles extends through the opening of the housing portion and is visible to a consumer.

18. The package of claim 13 wherein the article comprises at least a first pant of positioned within the housing portion such that the first pant extends through the opening, and such that at least a portion of a waistband region of the first pant is visible through the bag.

19. The package of claim 17 wherein the feature of the articles is selected from cloth-like material used to make the underwear, elastic waistbands, and back-label indicators.

20. The package of claim 13 further comprising a hole in the header strip enabling the package to be hung on a pin or rod on a display rack in a first shelf configuration.

21. The package of claim 13 wherein the first and second web comprises transparent materials.

22. The package of claim 13 wherein the first side portion and the second side portion are not attached.
